(19) United States
(12) Patent Application Publication
(10) Pub. No.: US 2003/0161851 A1
(43) Pub. Date: Aug. 28, 2003

(54) Cosmetic lip product with sour flavor

(75) Inventors: Paul J. Breha III, Seven Hills, OH (US); Ruth E. Higgs, North Olmsted, OH (US)

Correspondence Address: Richard J. Minnich, Esq., Fay, Sharpe, Fagan, Minnich & McKee, LLP, 7th Floor, 1100 Superior Avenue, Cleveland, OH, 44114-2518, US

(73) Assignee: Bonne Bell, Inc.

(21) Appl. No.: 10/337,127

(22) Filed: Jan. 6, 2003

Related U.S. Application Data
(63) Continuation-in-part of application No. 10/075,219, filed on Feb. 14, 2002.

(51) Int. Cl. A61K 7/025

(52) U.S. Cl. 424/401

(57) ABSTRACT

Methods for making a sour tasting cosmetic lip product containing one or more acids dissolved in a base composition, and the products formed thereby, are disclosed. The products possess a pleasing sour taste while not producing irritation or burning on the lips or mouth. Ingredients such as sunblocks, moisturizers and vitamins may be added to the compositions depending on the properties desired in the final product. The compositions according to the present invention comprise from about 2% to about 25% by weight acid and are preferably anhydrous.

This application is a continuation-in-part of U.S. application Ser. No. 10/075,219 filed Feb. 14, 2002.

### FIELD OF INVENTION

The present invention relates to cosmetic and therapeutic compositions for the lips containing one or more acids. More particularly, the present invention relates to lip compositions comprising an acid that exhibit a sour taste.

### BACKGROUND OF THE INVENTION

Unprotected skin is very susceptible to drying out and becoming irritated from exposure to the elements. This is especially true with regard to the lips, which have been found to be even more vulnerable to water loss than typical skin. This is due, in part, to the fact that the lips have a thinner stratum corneum, the outermost of three layers comprising skin, and contain a lesser amount of lipids than skin on other parts of the body. When the lipid barrier is depleted or inadequate, lips dry out, becoming irritated and prone to cracking. Lips are susceptible to other harms as well. For example, due to the fact that lips contain less melanin than other areas of skin, they are at risk of sunburn and UV damage.

In response to this, there have been various products introduced by manufacturers to keep the lips in a moisturized, smooth condition and protected from damage. These products most typically contain waxes or oils that mitigate the amount of moisture that escapes from the lips into the atmosphere. Some products may additionally contain moisturizers as well as healing agents to actually replace the moisture that has been lost and/or sooth damaged lips. Various sunscreens are also added to some products to protect the lips from sun damage.

Although useful, the taste and/or consistency of these products often does not appeal to consumers, especially younger users in their adolescent and teenage years. These users often want a tasteful and long lasting lip product that can easily be reapplied as desired. It has been found that slightly sour tasting candies and other products are pleasant to many consumers and in great demand. One only need consider the great variety of sour tasting candy and beverages now on the market to realize this. These products are typically given their sour taste by incorporating a small amount of acid, typically one or more fruit acids, into the product formulation. These acids stimulate taste receptors mainly along the sides of the tongue that are sensitive to such compounds. The concentration of acid in these products must be carefully controlled, great enough such that a pleasant “tanginess” is provided, but not so great that the acid burns or irritates the mouth.

The use of some types of acids in skin products is known. For example, it is known to use various alpha-hydroxy acids in skin lotions and creams to accelerate skin cell turnover by chemically exfoliating the top layer of skin cells. (See, e.g., U.S. Pat. No. 5,939,085). These products, however, are designed to achieve practically the exact opposite result of the present invention. Whereas the present invention is designed as a humectant and moisturizer and uses a relatively small amount of acid as a gentle flavoring agent, the hydroxy acid containing products are used to treat acne and other skin conditions by removing the cells from the skin surface using an astrigent acid-based composition, in effect “burning off” the upper skin layer. The use of such products on the delicate skin of the lips would be extremely discomforting.

With the great demand for sour tasting products, a need exists for a sour tasting lip product that can easily be reapplied, will not irritate the lips when used as directed, can be formulated with various lip moisturizers and protectors, and that is relatively stable over extended periods of time.

### SUMMARY OF THE INVENTION

In one aspect, the present invention provides a sour tasting lip product containing one or more acids dissolved in a base composition, the product being solid and capable of being applied in stick form at room temperature. The acids used in the invention are generally any of the naturally occurring fruit acids, although other acids may also be used.

In a second aspect, the present invention provides a method of forming a sour tasting lip product in stick form.

In a third aspect, the present invention provides a high melting point sour tasting lip product containing one or more acids dissolved in a base composition, having a melting point greater than about 60° C. (140° F.).

Anhydrous acids are first dissolved in a suitable solvent, such as propylene glycol, under heat and stirring. Preferably the solvent and other components of the composition are substantially anhydrous. As used herein, “substantially anhydrous” means containing less than about 5% water by weight. After the acid is fully dissolved, a suitable cosmetic lip base is added to the mixture. As used herein, “cosmetic base” refers to any cosmetically or pharmaceutically acceptable carrier or base composition suitable for use in lip glosses, lip balms, lipsticks, and other compositions appropriate for application on the lips and around mucous membranes. This base may contain any of a number of known ingredients, such as various oils, moisturizers, emollients, etc., in any concentration that does not adversely affect the properties of the final composition. Other components may also be added, such as vitamins, sweeteners and other flavorings, sunblock agents, colorants, etc. The final composition is mixed to form a homogenous composition of the desired consistency.

Depending on the type of flavoring added to the mixture, the final composition will typically have a tart, sour fruit taste. The content of the final composition is preferably about 2% to about 25% by weight acid, about 3% to about 25% solvent, about 1% to about 10% flavoring, and about 55% to about 85% cosmetic lip base. The final composition will preferably have a pH of about 2.00-4.00. Although the final composition may be formulated to have any consistency desired, in one embodiment, the lip composition produced by the present invention will typically have a consistency similar to that of a chapstick or LIP SMACKER® lip gloss. Therefore it may easily be stored in small cosmetic dispensers and applied to the lips in a similar manner.

### BRIEF DESCRIPTION OF THE DRAWINGS

FIGS. 1-3 are side perspective views of a container for the retail packaging of a sour lip product according to one embodiment of the invention.

FIG. 4 is a side perspective view of a sour lip product in stick form in a container according with a second embodiment of the present invention.

### DETAILED DESCRIPTION OF THE INVENTION

The present invention provides a process for producing sour tasting cosmetic lip products containing one or more acids dissolved in a cosmetic lip base composition, and the products formed from this process. Other ingredients may be incorporated into the lip products of the present invention, including, but not limited to, moisturizing aids, vitamins, and sunblocks.

The acids suitable for use in the present invention include any acid that will produce a stable mixture and a sour flavor when combined in specific concentrations with a cosmetic lip base composition, but, when combined in a final product, will not burn the lips or mouth even after heavy use. By “heavy use”, the applicants are generally referring to continuous extended exposure of 24 hours or greater and/or repeated applications within that time. Preferably, the acids used in the present invention include one or more of the acids naturally occurring in various fruits, including but not limited to citric acid, malic acid, tartaric acid, fumaric acid, propionic acid, acetic acid, and lactic acid. These acids are weaker than some of the stronger mineral acids, such as hydrochloric acid, and produce a pleasing sour taste, especially when combined with complementary fruit flavorings in the final composition. Therefore, although other acids are contemplated by the invention, fruit acids will be used in all subsequent discussion for clarity and convenience. Similarly, while the subsequent discussion may refer to a single acid, it must be remembered that any combination of one or more fruit acids may be used in formulating a composition according to the present invention. A preferred formulation of acids for use in the present invention is citric acid or a combination of citric acid with one or more other fruit acids. Preferably, the total amount of acid in the final product is from about 2% to about 25% by weight, more preferably about 2% to about 15% by weight, and most preferably from about 4.5% to about 5.5%. In the case of the use of multiple acids, the combined amount of all such acids will fall within these concentration ranges. The final composition will typically have a pH of about 2.00-4.00, and preferably about 2.68-3.90. In the case of an anhydrous final composition, pH is measured on a 5% by weight aqueous solution of the composition.

The acids for use in the present invention are preferably USP grade (99+% pure) and purchased in anhydrous powder form. These acids are available from various companies such as Aldrich Chemical Co., Milwaukee, Wis. To produce more uniform mixing and promote better consistency in the final composition, the acid is preferably dissolved in a solvent prior to mixing with a cosmetic lip base. Suitable solvents include any solvents in which the selected powder acid is soluble and which are themselves soluble in the cosmetic lip base. Preferable solvents include compounds that allow for dissolution of the relatively polar acids while being easily incorporated themselves into the generally non-polar cosmetic base. Thus, preferred solvents include, but are not limited to glycerin, propylene glycol, dipropylene glycol, butylene glycol and its higher homologues. In addition to being somewhat sweet and thus enhancing the flavor of the final composition, these solvents readily dissolve the acids upon heating and are easily incorporated into a typical cosmetic lip base. A most preferred solvent is propylene glycol. The use of water as a solvent, although workable and contemplated by the invention, is not preferred, since the reactivity and stability of the acid in the final composition becomes more difficult to control, which may lead to a burning sensation on the lips and mouth. Preferably, the total amount of solvent in the final product is from about 3% to about 25% by weight, more preferably about 5% to about 20% by weight.

Suitable cosmetic lip bases for the present invention may include any compositions useful in cosmetic lip products that are compatible with the acid and solvent used in formulating the invention. Cosmetic bases in the present invention may include various ingredients that find use in lip glosses, lip balms, lipsticks, lip moisturizers, and other lip products in any concentration that does not adversely affect the sour flavoring and other properties of the final composition. Thus, ingredients suitable for use in the cosmetic bases of the present invention may include, but are not limited to, various oils, waxes, humectants, emollients, proteins, preservatives, antioxidants, emulsifiers, sunblocks, colorants, fragrances, moisturizers, therapeutic agents and vitamins.

“Oils” as used herein encompass not only naturally occurring plant, animal and mineral oils, but also oil-like emollients such as fatty esters, fatty alcohols and silicone oils. Suitable moisturizers and oils include, but are not limited to, polybutene; lanolin; petrolatum; vegetable oil; mineral oil; castor oil, isopropyl palmitate; diisopropyl dimerate; glycerides, triglycerides and other esters; glycerols; diglycerols; olive oil; vitamin E acetate; and mixtures thereof.

“Waxes” as used herein encompass not only those plant, animal and mineral waxes containing esters of fatty acids and alcohols and saturated hydrocarbons, but also synthetic resins having a wax-like texture, such as silicone waxes. Suitable waxes include, but are not limited to, beeswax; paraffin wax; lanolin wax; jojoba wax; carnauba wax; spermaceti; ozokerite; candellila wax; animal wax; synthetic wax; plant and mineral waxes; and mixtures thereof.

Suitable therapeutic agents or other active ingredients suitable for use in the present invention include, but are not limited to, aloe vera; elastin; collagen; vitamin E and derivatives thereof; vitamin C and derivatives thereof; vitamin A and derivatives thereof; allantoin; calamine; dimethicone; cocoa butter; shark liver oil; botanical extracts; phospholipids; and mixtures thereof.

Other materials suitable for use in the present invention and specific formulations are disclosed in “Cosmetics: Science and Technology”, 2nd Ed., Vol. 1, Wiley lnterscience, 1972; and “The Chemistry and Manufacture of Cosmetics”, 2nd Ed., Vol. IV, chapter 44, M. G. DeNavarre, Continental Press, 1975, the disclosures of which are incorporated herein by reference.

Preferably, the cosmetic base will contain polybutene and mineral oil. The use of greater than negligible amounts of water in the cosmetic base, while contemplated, is not preferred, for the same reasons that water is not a preferred solvent for the acid, as stated above. Preferably, the total amount of cosmetic base in the final product is from about 55% to about 90% by weight, more preferably about 60% to about 85% by weight.

In addition to the acid, solvent, and cosmetic lip base, other ingredients may be added to the lip compositions of the present invention. These ingredients may be part of the cosmetic base, or they may be added separately. Typically, for ease of production, these ingredients are those components that are only desired in specific formulations of the present invention. The cosmetic base and other ingredients may be combined into a bulk base. In this way, only a single cosmetic base is needed, which can be used for all formulations, while those additional ingredients only desired in specific instances may easily be added or have their concentration changed without altering the cosmetic base. Representative ingredients that may typically be added to the composition separately from the cosmetic base include, but are not limited to, artificial and natural sweeteners, TiO₂, and artificial and natural flavorings.

In a preferred embodiment, an artificial sweetener or sugar by-product that exhibits a sweetness anywhere from 10 to 500 times greater than natural sugar is added to the composition as a flavor enhancer, along with one or more proprietary flavorings available from various sources. Although sugar may be used as a sweetener, an artificial sweetener is preferred since they generally exhibit better long-term stability and greater resistance to microbial buildup. A preferred sweetener for use in the present invention is “Flavor Enhancer”, available from Noville, Inc. Preferably, the amount of sweetener added is an amount necessary to impart, in conjunction with the acid, a pleasing sour taste to the final composition that is similar to various commercially available sour candies. Typically, depending on the sweetener, this may be from about 0.05% to about 2.00% by weight.

Proprietary flavorings, such as those for use in many foods and candies, are available from various manufacturers. Preferably, the total amount of proprietary flavoring in the final product is from about 1% to about 10% by weight, more preferably about 1% to about 7% by weight. TiO₂ functions as both a whitening agent and a sunblock and may be added in varying amounts, depending on the amount of whitening and SPF protection desired in the final formulation.

There are various methods to manufacture the compositions of the present invention. As stated earlier, the acid is preferably dissolved in a solvent prior to being combined with the cosmetic base. To begin the process, a production vessel is filled with solvent and the requisite amount of acid and additional ingredients as desired is added to it. The mixture is agitated or stirred to effectuate mixing until the acid and other components are completely dissolved in the solvent and forms a solution. Depending on the solvent, the solvent can be heated to facilitate dissolution of the acid. Typically, with the use of propylene glycol as the solvent, the solvent is heated to between about 80° and 90° C. and stirred for about 30 to 60 minutes. Once the acid is completely dissolved, the cosmetic lip base and additional ingredients that are to be incorporated into the final composition are added to the solution. Preferably, the cosmetic base is added first and mixed thoroughly followed by the other ingredients. Depending on one's perspective, the solution can be thought of as being dissolved in the cosmetic base, or vice versa. Both expressions are synonymous as used herein, and refer to the combining of the two materials to form a substantially homogeneous mixture. The mixture is then stirred to completely dissolve the components and create a homogenous final composition. Depending on the actual ingredients used in a specific formulation, this composition may be a high quality suspension.

Depending on the exact composition of the cosmetic base and the concentration of ingredients, the final composition may have a consistency of anything from a conventional lipstick or hard wax to a thin cream. In a first embodiment, a typical composition according to the present invention will have a consistency similar to petrolatum or a thick cream with a viscosity of about 19560 centipoise as measured using a No. 4 spindle at 30 rpm and 25° C. and a specific gravity of about 0.88-0.92 g/ml. In this respect, a “doe-foot” applicator is useful in applying the composition. This type of applicator consists of a small absorbent tip on the end of a thin tubular body. The absorbent tip is dipped in the composition and then dabbed on the lips, transporting the composition from the tip to the lips.

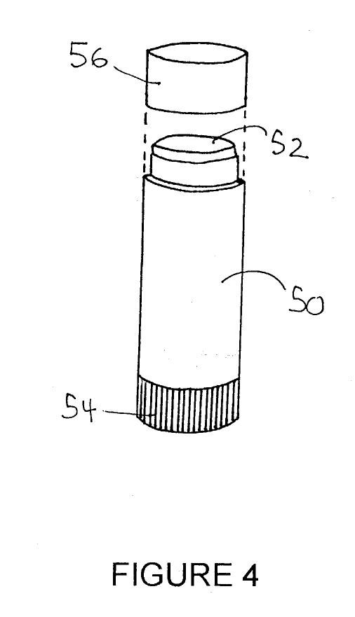

In a second embodiment, a lower concentration of cosmetic base may be used with an additional wax component. In this embodiment, the composition will be a solid at room temperature (25° C.) with the consistency of a conventional lipstick. In this embodiment, the composition may be packaged in stick form like a lipstick. An example of a packaged product incorporating this second embodiment of the present invention can be seen in FIG. 4. The packaged article includes a tubular body member 50 containing a solid stick of the sour lip product 52. The tubular body member 50 is attached to a base 54, The base 54 is rotatable about a longitudinal axis of the body member 50 as is known in the art for advancing the sour lip product stick 52. A cap 56 can be mounted on the body member 56 to prevent the product 52 from inadvertently contacting clothing, handbag interiors, etc.

In a third embodiment, a higher melting point cosmetic base may be used to produce a higher melting point composition with a melting point greater than about 60° C. (140° F.), preferably about 65-75° C. In this embodiment, the composition can be packaged as a thick creamy lip treatment to be applied with the finger.

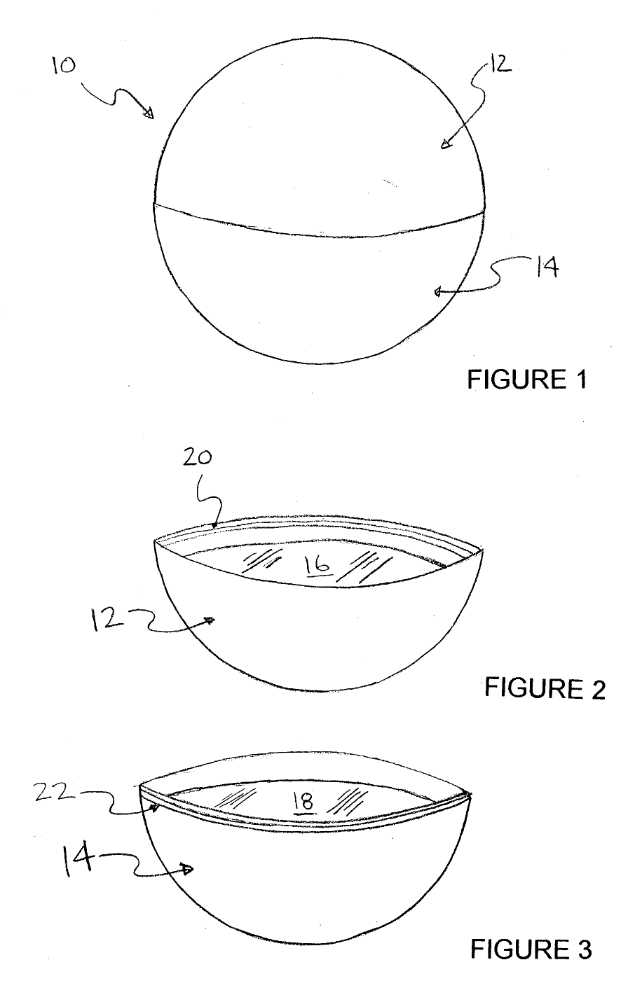

An example of a packaged product incorporating this third embodiment of the present invention can be seen in FIGS. 1-3. The packaged article comprises a spherical container 10 and includes selectively separable first and second halves 12, 14. The first half contains a conventional sweet lip product 16, as is known in the art, such as a sweet lip balm. Such products are available under various trade names from various suppliers, such as “Lip Smackers” available from Bonne Bell Inc. The second half 14 contains a sour lip product 18 according to the present invention. The spherical shape provides a small convenient case for the product that prevents accidental spilling or contaminating of the contents while also allowing the container to easily fit in pockets, purses, etc. without catching or snagging on clothing or other accessories carried by the consumer. Together, the two halves give consumers the option of applying a sweet lip product or a sour lip product at any given time, depending on their preference. In addition, the two products can be applied concurrently, providing a pleasing “sweet and sour” taste. The packaged article 10 is provided with a means for selectively attaching the first half 12 to the second half 14. As shown in FIGS. 2 and 3, this means can include screw threads 20, 22 on the two halves. Of course, other attaching means are also contemplated, such as a snap closure, hinged closure, velcro, buttons, etc. In addition, other container shapes are also contemplated.

Although colorants may be added to the composition as described above to give the composition any color desired, added TiO₂ will give the composition an opaque white color that applies as a substantially clear layer on the lips in the absence of any added colorants.

### EXAMPLES

The following examples are presented for the purpose of further illustrating the nature and scope of the present invention and are not intended as a limitation of the scope thereof. It should be appreciated that the present invention is in no way restricted to the following examples.

Formulation 1

The formulation in table 1 lists the concentrations of the various ingredients, except for the flavor and a solubilizing agent, used to produce a bulk base for the samples produced according to Examples 1-6. The following formulation may be used no matter what flavor of product is being manufactured. The concentrations are in weight percent.

TABLE 1
Bulk Base Formulations for Samples in Examples 1-6

| | Concentration | |
|---|---|---|
| Ingredient | (% w/w) | Supplier |
| Propylene Glycol USP | 7.00 | Vopak |
| Cosmetic Base¹ | 81.81 | Bonne Bell, Inc. |
| White Beeswax | 2.75 | Strahl & Pitsch |
| Citric Acid USP | 5.53 | Vopak |
| Sodium saccharine (20% | 0.80 | Bonne Bell, Inc. |
| solution in propylene glycol) | | |
| Flavor Enhancer AN120810 | 0.30 | Noville Inc. |
| Vitamin E Acetate | 0.50 | BASF |
| BV-OSC² | 0.01 | Barnet |
| TiO₂ (37% in mineral oil) | 0.10 | Bonne Bell, Inc. |
| Timica 1500³ | 1.20 | RONA |
| Lecinol S-10⁴ | 0.30 | Barnet |
| Total | 100.00% | |

¹Contains: petrolatum (m.p. 45° C.), polybutene, mineral oil, coconut oil, ozokerite, glycerin, trioctyldodecyl citrate, cetyl ricinoleate, shea butter extract, hydrogenated castor oil, mica, sorbitol, paraffin, propyl gallate, silica, propylpareben, butylparaben, aloe extract, ethylhexyl methoxycinnamate, tocopheryl acetate, tetrahexyldecyl ascorbate and propylene glycol dipelargonate.
²Vitamin C in an oil solution
³Pearling agent
⁴Lecithin derived stabilizer

The above-formulated composition may be considered a bulk base since, as stated, it may be used with any of the various flavored examples 1-6. Formulations of a final lip composition were produced using various flavors. These formulations are listed in Table 2 as examples 1-6.

TABLE 2
Final Lip Composition Formulations

| | | Concentration (% w/w) | | | | | |
|---|---|---|---|---|---|---|---|
| Ingredient | Supplier | 1 | 2 | 3 | 4 | 5 | 6 |
| Bulk Base | applicant | 93.80 | 93.80 | 94.25 | 95.05 | 94.30 | 94.05 |
| Grape flavor | FONA, | | | | | | 4.75 |
| | #856.193/WC | | | | | | |
| Rasberry/Cherry | Bell Flavors & | | | | 0.75 | 4.50 | |
| flavor | Fragrances | | | | | | |
| Cotton Candy flavor | Wessel | | 5.00 | | 2.00 | | |
| Strawberry flavor | Arylessence | | | | 1.00 | | |
| Watermelon flavor | J. Manheimer | | | 4.55 | | | |
| Lemonade flavor | Custom Essence, | 5.00 | | | | | |
| | CE14019 | | | | | | |
| Solubilisant LRI¹ | Costec/LCW | 1.20 | 1.20 | 1.20 | 1.20 | 1.20 | 1.20 |

¹Solubilizing agent for stabilizing frangrances

Obviously, the concentrations provided for the various ingredients of the bulk base in table 1 will change when combined with the flavor and Solubilisant LRI in the final composition. The new concentrations can be easily calculated. For example, the citric acid, which is present in a concentration of 5.53% by weight in the bulk base prior to the addition of flavor and solubilizing agent, will be present in a concentration of 5.19% by weight in a final composition formulated according to sample 1 (5.53×0.938=5.19).

A reaction vessel is filled with the amount of propylene glycol necessary to produce the specified concentration in the final composition. To this vessel, the acid, white beeswax and Lecinol S-10 are added in the specified amounts. The resulting mixture is heated to between about 800 and 90° C. with continuous stirring or similar agitation for about 30 to about 60 minutes until the acid and beeswax is completely dissolved and a liquid solution is achieved. Once the acid and wax are completely dissolved, the mixture is allowed to cool slightly to about 75 to 80° C. With the propylene glycol/acid/wax/Lecinol S-10 solution maintained at a temperature of between 75° and 80° C., the cosmetic lip base, which has been preheated to about 80 to 85° C., is added to the vessel with continued mixing. Once the cosmetic base has been added, the resultant mixture is maintained at a temperature of about 80° C. with continuous mixing. The heat is then removed and, with continued stirring, the mixture is allowed to cool. When the mixture reaches a temperature below about 75° C., the other ingredients listed in table 1 above are added to the mixture. The mixture is then stirred for an additional 1 to 4 hours until the mixture begins to thicken to the desired consistency as it continues to cool. The mixture is covered and allowed to sit at room temperature for about 6 to 24 hours. The mixture is then heated to about 65° C. with continuous stirring for approximately 1 to 3 hours to achieve a final smooth consistency. The bulk base product has an opaque white color and a consistency similar to a skin cream. This final bulk base may then be used to produce any flavor final lip composition desired.

To produce the final composition, the Solubilisant LRI is heated in a separate tank to about 50° C. Once it is thoroughly heated, the flavor is added. This mixture is stirred thoroughly until it is a homogenous composition. The mixture is then added to the requisite amount of bulk base in a separate tank. The resulting composition is mixed for approximately 1.5 hours or until homogenous.

Examples 1-6 above will produce a final composition having a consistency of a thick cream at room temperature and having a melting point of about 39-42° C. according to a first embodiment of the invention as described above, which may be conveniently packaged in small vials and is most easily applied to the lips using a “doe-foot” or similar applicator as earlier stated.

Formulation 2

The formulation in table 3 lists the concentrations of the various ingredients, except for the flavor and a solubilizing agent, used to produce a bulk base for the samples produced according to Examples 7-12. The following formulation may be used no matter what flavor of product is being manufactured. The concentrations are in weight percent.

TABLE 3
Bulk Base Formulations for Samples in Examples 7-12

| | Concentration | |
|---|---|---|
| Ingredient | (% w/w) | Supplier |
| Propylene Glycol USP | 11.00 | Vopak |
| Cosmetic Base¹ | 66.75 | Bonne Bell, Inc. |
| White Beeswax | 2.75 | Strahl & Pitsch |
| Citric Acid USP | 5.53 | Vopak |
| Paraffin Wax | 6.71 | Strahl & Pitsch |
| (60-63° C. m.p.) | | |
| Stearic Acid | 1.00 | Proctor & Gamble |
| Ozokerite Wax | 1.50 | Strahl & Pitsch |
| (82-86° C. m.p.) | | |
| Sodium saccharine (20% | 0.80 | Bonne Bell, Inc. |
| solution in propylene glycol) | | |
| Flavor Enhancer AN120810 | 0.30 | Noville Inc. |
| Vitamin E Acetate | 0.50 | BASF |
| BV-OSC² | 0.01 | Barnet |
| BOIS II³ | 2.00 | Barnet |
| TiO₂ (37% in mineral oil) | 0.10 | Bonne Bell, Inc. |
| Timica Sparkle 1500⁴ | 1.20 | Engelhard |
| Lecinol S-10⁵ | 0.30 | Laurichem |
| Total | 100.00% | |

¹Contains: petrolatum, polybutene (m.p. 45° C.), mineral oil, coconut oil, ozokerite, glycerin, trioctyldodecyl citrate, cetyl ricinoleate, shea butter extract, hydrogenated castor oil, mica, sorbitol, paraffin, propyl gallate, silica, propylpareben, butylparaben, aloe extract, ethylhexyl methoxycinnamate, tocopheryl acetate, tetrahexyldecyl ascorbate and propylene glycol dipelargonate.
²Vitamin C in an oil solution
³2% Bois oil (containing sandlewood, barley and phillodendron extract)
⁴Pearling agent
⁵Lecithin derived stabilizer

The above-formulated composition may be considered a bulk base since, as stated, it may be used with any of the various flavored examples 7-12. Formulations of a final lip composition were produced using various flavors. These formulations are listed in Table 4 as examples 7-12.

TABLE 4
Final Lip Composition Formulations (Examples 7-12)

| | | Concentration (% w/w) | | | | | |
|---|---|---|---|---|---|---|---|
| Ingredient | Supplier | 7 | 8 | 9 | 10 | 11 | 12 |
| Bulk Base | applicant | 95.00 | 95.00 | 95.45 | 96.25 | 95.50 | 95.25 |
| Grape flavor | FONA, | | | | | | 4.75 |
| | #856.193/WC | | | | | | |
| Rasberry/Cherry | Bell Flavors & | | | | 0.75 | 4.50 | |
| flavor | Fragrances | | | | | | |
| Cotton Candy flavor | Wessel | | 5.00 | | 2.00 | | |
| Strawberry flavor | Arylessence | | | | 1.00 | | |
| Watermelon flavor | J. Manheimer | | | 4.55 | | | |
| Lemonade flavor | Custom Essence, | 5.00 | | | | | |
| | CE14019 | | | | | | |

Obviously, the concentrations provided for the various ingredients of the bulk base in table 3 will change when combined with the flavor in the final composition. The new concentrations can be easily calculated. For example, the citric acid, which is present in a concentration of 5.53% by weight in the bulk base prior to the addition of flavor, will be present in a concentration of 5.25% by weight in a final composition formulated according to sample 1 (5.53×0.950=5.25).

A reaction vessel is filled with the amount of propylene glycol necessary to produce the specified concentration in the final composition. To this vessel, the acid, beeswax, lecinol S-10 and stearic acid are added in the specified amounts. The resulting mixture is heated to about 90° C. with continuous stirring or similar agitation for about 30 to about 60 minutes until the acid is completely dissolved and a liquid solution is achieved. Once the components are completely dissolved, the cosmetic lip base is added to the vessel with continued mixing while maintaining a batch temperature of about 90° C. Once the cosmetic base has been added, the paraffin and ozokerite are added with agitation. Once the waxes have melted, the mixture is allowed to cool slightly. When the mixture reaches about 75-80° C., the sodium saccharin, flavor enhancer, vitamin E, Timica 1500, titanium dioxide, Bois II and BV-OSC are all added. The mixture is continuously stirred while it is allowed to cool to about 65-70° C. The flavors are then added with continuous mixing as called for by the flavor being produced according to table 4. When the mixture is homogenous, it is transferred to filling equipment and filled at 55-60° C.

The formulations made according to examples 7-12 will have a melting point of about 47° C. and generally have a more waxy consistency than formulation 1, similar to that of a conventional lipstick. In such a case, compositions according to a second embodiment of the invention are most conveniently packaged in a lipstick type case or a similar manner as described above. This differences in consistency and melting point as compared to the first embodiment is due in part to the use of a smaller amount of cosmetic base and the addition of paraffin and ozokerite wax to the bulk base set forth in table 3. Preferably, the paraffin will be present in a concentration of about 5% to about 10% by weight. Depending on the exact amount, the composition will typically have a melting point of from about 40° C. to about 50° C.

Formulation 3

The formulation in table 5 lists the concentrations of the various ingredients, except for the flavor and a solubilizing agent, used to produce a bulk base for the samples produced according to Examples 13-18. The following formulation may be used no matter what flavor of product is being manufactured. The concentrations are in weight percent.

TABLE 5
Bulk Base Formulations for Samples in Examples 13-18

| | Concentration | |
|---|---|---|
| Ingredient | (% w/w) | Supplier |
| Propylene Glycol USP | 7.00 | Vopak |
| Cosmetic Base¹ | 81.96 | Bonne Bell, Inc. |
| White Beeswax | 2.75 | Strahl & Pitsch |
| Citric Acid USP | 5.53 | Vopak |
| Sodium saccharine (20% | 0.80 | Bonne Bell, Inc. |
| solution in propylene glycol) | | |
| Flavor Enhancer AN120810 | 0.30 | Noville Inc. |
| Vitamin E Acetate | 0.50 | BASF |
| BV-OSC² | 0.01 | Barnet |
| TiO₂ (37% in mineral oil) | 0.10 | Bonne Bell, Inc. |
| Timica Sparkle 1500³ | 0.75 | Engelhard |
| Lecinol S-10⁴ | 0.30 | Laurichem |
| Total | 100.00% | |

¹Contains: high melting point petrolatum (m.p. 77° C.), polybutene, mineral oil, coconut oil, ozokerite, glycerin, trioctyldodecyl citrate, cetyl ricinoleate, shea butter extract, hydrogenated castor oil, mica, sorbitol, paraffin, propyl gallate, silica, propylpareben, butylparaben, aloe extract, ethylhexyl methoxycinnamate, tocopheryl acetate, tetrahexyldecyl ascorbate and propylene glycol dipelargonate.
²Vitamin C in an oil solution
³Pearling agent
⁴Lecithin derived stabilizer

The above-formulated composition may be considered a bulk base since, as stated, it may be used with any of the various flavored examples 13-18. Formulations of a final lip composition were produced using various flavors. These formulations are listed in Table 6 as examples 13-18.

TABLE 6
Final Lip Composition Formulations (Examples 13-18)

| | | Concentration (% w/w) | | | | | |
|---|---|---|---|---|---|---|---|
| Ingredient | Supplier | 13 | 14 | 15 | 16 | 17 | 18 |
| Bulk Base | applicant | 95.00 | 95.00 | 95.45 | 96.25 | 95.50 | 95.25 |
| Grape flavor | FONA, | | | | | | 4.75 |
| | #856.193/WC | | | | | | |
| Rasberry/Cherry | Bell Flavors & | | | | 0.75 | 4.50 | |
| flavor | Fragrances | | | | | | |
| Cotton Candy flavor | Wessel | | 5.00 | | 2.00 | | |
| Strawberry flavor | Arylessence | | | | 1.00 | | |
| Watermelon flavor | J. Manheimer | | | 4.55 | | | |
| Lemonade flavor | Custom Essence, | 5.00 | | | | | |
| | CE14019 | | | | | | |

Obviously, the concentrations provided for the various ingredients of the bulk base in table 6 will change when combined with the flavors in the final composition. The new concentrations can be easily calculated. For example, the citric acid, which is present in a concentration of 5.53% by weight in the bulk base prior to the addition of flavor will be present in a concentration of 5.25% by weight in a final composition formulated according to sample 13 (5.53×0.95=5.25). The method of making the examples of formulation 3 was similar to that used in making the examples of formulation 1 with the exception of the removal of the solubilisant LRI.

The formulations made according to examples 13-18 may have a consistency similar to petroleum jelly and will generally exhibit a higher melting point (m.p. of about 68-70° C.) compared to those formulations made according to examples 1-12. This is due, in part, to the use of a higher melting point petrolatum in the cosmetic base. Preferably, the melting point of the petrolatum is greater than about 60° C., more preferably between about 75° C. and 85° C. so as to produce a final product having a melting point of from about 65-75° C. (150° F.-167° F.). Formulations made according to this embodiment will have the consistency of a very thick cream (generally a stiffer consistency than those made according to formulation 1) and can be packaged in any suitable manner. One exemplary method for packaging this formulation is together with a sweet lip product in a spherical container as shown in FIGS. 1-3 and discussed above.

The invention has been described with reference to various preferred embodiments. Modifications and alterations will occur to others upon a reading and understanding of the specification. The invention is intended to include all such modifications and alterations insofar as they come within the scope of the appended claims and the equivalents thereof.

What is claimed is:

1. A composition for topical application on the lips, said composition comprising:
a cosmetic base including a petrolatum having a melting point greater than about 60° C.; and
an acid present in a concentration sufficient to produce a sour taste when said composition is applied to the lips without discomfort or irritation.

2. A composition according to claim 1, further including a solvent that is miscible in said cosmetic base and in which said acid is miscible.

3. A composition according to claim 2, wherein said solvent is propylene glycol.

4. A composition according to claim 1, wherein said composition has a melting point of between 65° C. and 75° C.

5. A composition according to claim 1, wherein said cosmetic base has a melting point of between 75° C. and 85° C.

6. A composition according to claim 1, wherein said composition exhibits a pH of about 2.00-4.00.

7. A composition according to claim 6, wherein said composition exhibits a pH of about 2.68- 3.90.

8. A composition according to claim 1, wherein said acid is present in a concentration of from about 2% to about 15% by weight of said total composition.

9. A composition according to claim 8, wherein said acid is present in a concentration of from about 4.5% to about 5.5% by weight of said total composition.

10. A composition according to claim 1, wherein said acid is one or more acids selected from the group consisting of citric acid, malic acid, tartaric acid, fumaric acid, propionic acid, acetic acid and lactic acid.

11. A composition according to claim 10, wherein said acid is citric acid.

12. A composition according to claim 1, wherein said composition further comprises a sweetener and a flavoring agent.

13. A composition according to claim 12, wherein said flavoring agent is a fruit or candy flavored composition.

14. A composition according to claim 12, wherein said sweetening agent is a concentrated sweetener that exhibits a sweetness from 10 to 500 times greater than natural sugar.

15. A composition according to claim 1, wherein said composition includes one or more additives selected from the group consisting of oils, waxes, humectants, emollients, preservatives, antioxidants, emulsifiers, colorants, sunblocks, moisturizers, healing agents and vitamins.

16. A composition according to claim 1, wherein said cosmetic base includes polybutene and mineral oil.

17. A composition according to claim 1, wherein said cosmetic base is present in a concentration of from about 60% to about 85% by weight of said total composition.

18. A sour tasting composition for application on the lips, said composition comprising:
one or more acids selected from the group consisting of citric acid, malic acid, tartaric acid, fumaric acid, propionic acid, acetic acid and lactic acid in a concentration of from about 2% to about 15% by weight of said total composition;
a solvent that is miscible in said cosmetic base and in which said acid is miscible in a concentration of from about 5% to about 20% by weight of said total composition; and
a cosmetic base in a concentration of from about 60% to about 85% by weight of said total composition including a petrolatum having a melting point greater than 60° C.

19. A composition for topical application on the lips and mouth area, said composition comprising:
a cosmetic base including stearic acid and a paraffin wax; and
one or more acids selected from the group consisting of citric acid, malic acid, tartaric acid, fumaric acid, propionic acid, acetic acid and lactic acid in a concentration of from about 2% to about 15% by weight of said total composition, wherein said wax is present in a concentration such as to impart a lipstick consistency to said composition and to permit said composition to be packaged in stick form.

20. A composition according to claim 19, further comprising bois oil.

21. A composition according to claim 19, wherein said paraffin wax is present in a concentration of from about 5 to about 10% by weight of said total composition.

22. A composition according to claim 19, wherein said composition has a melting point of from about 40° C. to about 50° C.

23. A composition according to claim 19, further including a solvent that is miscible in said cosmetic base.

24. A composition according to claim 19, wherein said composition exhibits a pH of about 2.00-4.00

25. A process for forming a composition suitable for application on the lips, said process comprising the steps of:
providing one or more acids selected from the group consisting of citric acid, malic acid, tartaric acid, fumaric acid, propionic acid, acetic acid and lactic acid;
providing beeswax and stearic acid, dissolving said acids, beeswax and stearic acid in a solvent at a temperature of about 90° C. with continuous stirring to produce a solution, said solvent selected from the group consisting of propylene glycol, dipropylene glycol, butylene glycol, ethylene glycol, and glycerin;
adding a cosmetic base to said solution with continuous stirring to form a mixture;
adding paraffin and ozokerite wax to said mixture;
cooling said mixture to about 65° C. to about 70° C.;
adding a flavoring agent to said mixture, and
forming said mixture into a stick form.

26. A product formed according to claim 25.

27. A lip product comprising a container including a first half containing a sweet flavored lip composition and a second half containing a sour flavored lip composition comprising a cosmetic base and one or more acids selected from the group consisting of citric acid, malic acid, tartaric acid, fumaric acid, propionic acid, acetic acid and lactic acid, wherein said first half and said second half are selectively separable.

28. A lip product according to claim 27, wherein said container is spherical and wherein said first half and said second half are hemispherical.

29. A lip product according to claim 27, wherein said first half and said second half are selectively connectable via mating screw threads.
